(19) United States
(12) Patent Application Publication
(10) Pub. No.: US 2020/0360235 A1
(43) Pub. Date: Nov. 19, 2020

(54) A VALVE FOR AN INJECTION DEVICE AND AN INJECTION DEVICE WITH SUCH A VALVE

(71) Applicant: Subcuject ApS, Hellebæk (DK)

(72) Inventor: Claus Schmidt MØLLER, Fredensborg (DK)

(21) Appl. No.: 16/964,306

(22) PCT Filed: Jan. 28, 2019
(86) PCT No.: PCT/DK2019/050034

§ 371 Date: Jul. 23, 2020

(30) Foreign Application Priority Data
Jan. 30, 2018 (DK) PA 2018 00049
Feb. 21, 2018 (DK) PA 2018 70111

(51) Int. Cl. A61J 1/20; A61M 5/31; A61M 5/28; A61M 5/172

(52) U.S. Cl. CPC A61J 1/2062; A61J 1/2089; A61J 1/2006; A61M 2005/312; A61M 5/288; A61M 5/172; A61M 2005/3103; A61M 5/3134

(57) ABSTRACT

A valve (116, 216) for an injection device (100) is disclosed, which valve is arranged to provide a fluid communication between a drug-filled container (102, 202) and a drug distribution system (101) for distributing a drug to an injection site during use of the device, wherein the valve is substantially cylindrically shaped and is arranged to be at least partly positioned within an outlet portion (123, 223) of the container so that, in an initial configuration, the valve tightly closes the outlet portion, wherein the valve is arranged to be moved through a first distance in a first direction partly into the con-tainer, whereby fluid communication is established through the outlet portion of the container, and wherein the valve is further arranged to be moved through a second distance in a second direction opposite of the first direction. Furthermore, an injection device (100) comprising such a valve (116, 126) is disclosed.

The invention relates to a valve for an injection device for subcutaneous injection of a therapeutic drug, wherein the valve provides a fluid pathway between a drug-filled container and a drug distribution system for distributing the drug to the injection site during use of the device. Furthermore, the invention relates to an injection device comprising such a valve.

### BACKGROUND OF THE INVENTION

During the recent years, self-injection of various therapeutic medicaments by means of auto injections or wearable injection devices on a daily, weekly, monthly or just on a one-time basis has become more common. These devices are normally disposable and configured to be used only once, and production cost and environmental impact have to be considered. As the users of these types of devices are not necessarily injecting themselves on a regular basis, the devices must be intuitive and safe to use, e.g., with automated needle retraction, and it must be clear for the user, for instance, when the injection is completed and the device can be removed. For many of the devices on the market, especially when it comes to wearable injection devices, injection force, needle retraction, signals etc. are controlled by means of electronically components.

WO 2017/129191 discloses a wearable injection device using the movement of the plunger to retract the needle after the injection. During activation of the device, a hollow needle is pushed through a septum on a cartridge by a movable part and, at the same time, a hypodermic needle is inserted in the patient and locked in position by the movable part. At the end of the injection, the hollow needle is pushed a distance out of the cartridge and, as this movement is transferred to the movable part, the hypodermic needle is set free to be retracted by a spring. A disadvantage of this configuration is that only cartridges with a septum can be used in the device, while part of the industry prefers a solution that allows syringes without a needle to be used in the device. Another disadvantage is that particles from the septum may be introduced to the drug when the needle penetrates the septum.

### BRIEF DESCRIPTION OF THE INVENTION

It is an objective of the present invention to provide a valve for an injection device and, thereby, an injection device without the above-described disadvantages of devices known in the art.

The present invention relates to a valve for an injection device, which valve is arranged to provide a fluid communication between a drug-filled container and a drug distribution system for distributing a drug to an injection site during use of the device, wherein the valve is substantially cylindrically shaped and is arranged to be at least partly positioned within an outlet portion of the container so that, in an initial configuration, the valve tightly closes the outlet portion, wherein the valve is arranged to be moved through a first distance in a first direction partly into the container, whereby fluid communication is established through the outlet portion of the container, and wherein the valve is further arranged to be moved through a second distance in a second direction opposite of the first direction.

In an embodiment of the invention, the fluid communication through the outlet portion is established at least partly by means of internal fluid channels through the valve.

In an embodiment of the invention, the fluid communication through the outlet portion is established at least partly along an outer surface of the valve.

In an embodiment of the invention, the fluid communication through the outlet portion is established at least partly by means of external and open fluid channels arranged on the outside of the valve.

The use of such a valve enables for obtaining an injection device, which does not comprise a needle for penetrating a septum of the drug-filled container, whereby is provided a bigger surface that is more suited for being pushed by a rubber plunger than a needle tip.

In an aspect of the invention, it relates to an injection device for subcutaneous injection of a therapeutic drug comprising a drug-filled container defining a centre axis and comprising an outlet portion and a movable plunger, a distribution system for distributing the drug to an injection site comprising a connecting part movable along the centre axis, and a valve as described above, which is at least partly positioned in the outlet portion of the container, wherein, in an initial configuration, the valve tightly closes the outlet portion of the container, wherein, during activation of the device, the connecting part causes the valve to move through a first distance in a first direction partly into the container, whereby fluid communication is established through the outlet portion of the container between the container and the distribution system, and wherein, at the completion of the injection, the plunger in the container pushes the valve through a second distance in a second direction opposite of the first direction, thereby moving the connecting part through the same second distance.

By letting the connecting part move at the completion of the injection, a means for actuating actions in the end of the injection such as, e.g., needle retraction is provided.

In an embodiment of the invention, the second distance is shorter than the first distance.

Such a configuration ensures that three different positions of the connecting part can be obtained, which can be used for tactile indications of the operational statuses of the device.

In an embodiment of the invention, the distribution system further comprises a delivery part with a hypodermic needle for insertion at an injection site at activation of the device, and a spring means compressed during the insertion, wherein the connecting part is arranged to maintain the delivery part in an exposed position in relation to the injection device during the injection, and wherein the connecting part is arranged to release the delivery part to be moved back into a retracted position within the injection device by the compressed spring means, when the connecting part is moved by the valve at the completion of the injection.

Thereby a very simple retraction mechanism for a wearable injection device is provided.

In an embodiment of the invention, the movement of the connecting part at the completion of the injection is electronically detected.

By adding an electronic unit that detects the movement of the connecting part, data regarding the injection can be stored and/or transmitted, e.g., to health care personnel.

In an embodiment of the invention, in the initial configuration, the valve is sealed by means of a breakable or removable seal.

By sealing the device in this way, a very simple and inexpensive sterile barrier for one-time use is provided.

In an embodiment of the invention, during activation of the device, the connecting part and the outlet portion of the container are sealed to each other.

By sealing the connecting part directly to the outlet portion of the container, the valve does not have to seal to the drug-filled container during injection. Instead of arranging a sealing directly between the connecting part and the container during the injection, a sealing may also be arranged directly between the connecting part and the valve.

### THE DRAWINGS

In the following, a few exemplary embodiments of the invention are described in further details with reference to the drawings, of which

FIG. 1 is a perspective view of a wearable injection device according to an embodiment of the invention,

FIG. 2 is an exploded view of a distribution system of a wearable injection device according to an embodiment of the invention,

FIG. 3 is a perspective view of a drug-filled container and a distribution system of an injection device in communication with each other through a valve according to an embodiment of the invention in a first state,

FIG. 4 is a perspective view of the same drug-filled container and distribution system in a second state,

FIG. 5 shows a perspective view of the same drug-filled container and distribution system in a third state,

FIG. 6 is a schematic cross-sectional view of a drug-filled container, a valve according to a first embodiment of the invention and a connecting part in a first state,

FIG. 7 is a schematic cross-sectional view of the same container, valve and connecting part in a second state,

FIG. 8 is a schematic cross-sectional view of the same container, valve and connecting part in a third state,

FIG. 9 is a schematic cross-sectional view of the same container, valve and connecting part in a fourth state,

FIG. 10 is a schematic cross-sectional view of a drug-filled syringe, a valve according to a second embodiment of the invention and a connecting part in a first state,

FIG. 11 is a schematic cross-sectional view of the same syringe, valve and connecting part in a second state,

FIG. 12 is a schematic cross-sectional view of the same syringe, valve and connecting part in a third state,

FIG. 13 is a schematic cross-sectional view of the same syringe, valve and connecting part in a fourth state,

FIG. 14 is a schematic cross-sectional view of a drug-filled syringe, a valve according to a third embodiment of the invention and a connecting part in a first state,

FIG. 15 is a perspective view of the valve from FIG. 14,

FIG. 16 is a schematic cross-sectional view of the syringe, valve and connecting part from FIG. 14 in a second state,

FIG. 17 is a schematic cross-sectional view of the same syringe, valve and connecting part in a third state, and

FIG. 18 is a schematic cross-sectional view of the same syringe, valve and connecting part in a fourth state.

### DETAILED DESCRIPTION OF THE INVENTION

In the following, the terms “up”, “down” and “downward” refer to the drawings and do not necessarily correspond to a situation of use.

Functional units, which are described and shown to comprise a number of parts fixed together and acting as one part, may be configured as one single part or comprise a more or less separate parts divided in a different way than described and shown without falling outside the scope of the invention.

The term “drug-filled container” 102 or just “container” covers all kinds of vessels and containers, from which a drug can be pressed out by means of a movable plunger 124, including all kinds of cartridges and syringes 202 made from glass or polymer.

By the term “actuator” is meant a drive mechanism that can deliver the necessary force to drive the drug out of the container 102, 202. This drive mechanism can be configured as a mechanical, hydraulic, chemical or electronic mechanism.

The term “distribution system” 101 covers all mechanisms, which by actuation of the device 100 can direct the drug from the drug-filled container 102, 202 to the injection site. This also includes multiple needle- or catheter-based embodiments.

The term “drug” is to be understood as biologically or small molecule medicaments to be inserted in an individual to cure a disease, for which the medicament is intended.

Only the parts of a wearable injection device 100, which are necessary to understand the function of the valve 116, 216, are described in the following and, hence, a fully equipped wearable injection device 100 is not described.

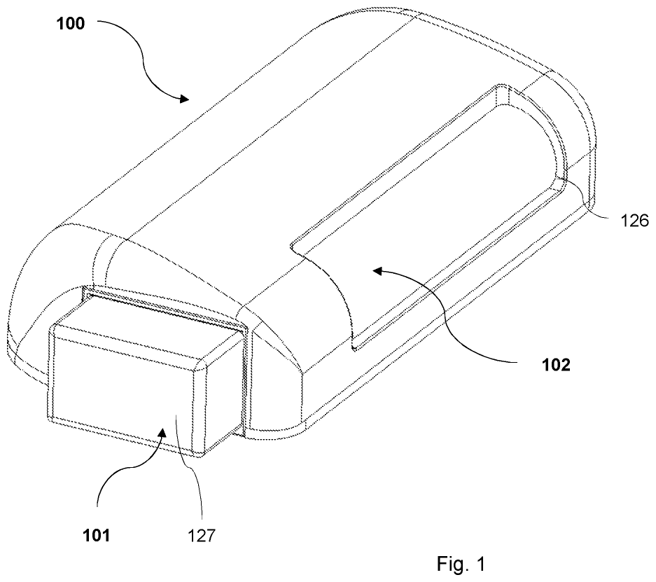

FIG. 1 is a perspective view of a wearable injection device 100 according to an embodiment of the invention. A drug-filled container 102 can be seen through an opening 126 in the outer housing parts. The push button 127, which forms part of a distribution system 101, is preferably made of a soft and flexible material and acts upon one or more of the inner parts (not shown in this drawing) of the distribution system 101, when it is pushed. The illustrated device 100 is a preferred device 100 to be used with the valve 116 of the invention, but the valve 116 can also be configured for being used in other types of injection devices, such as auto injections.

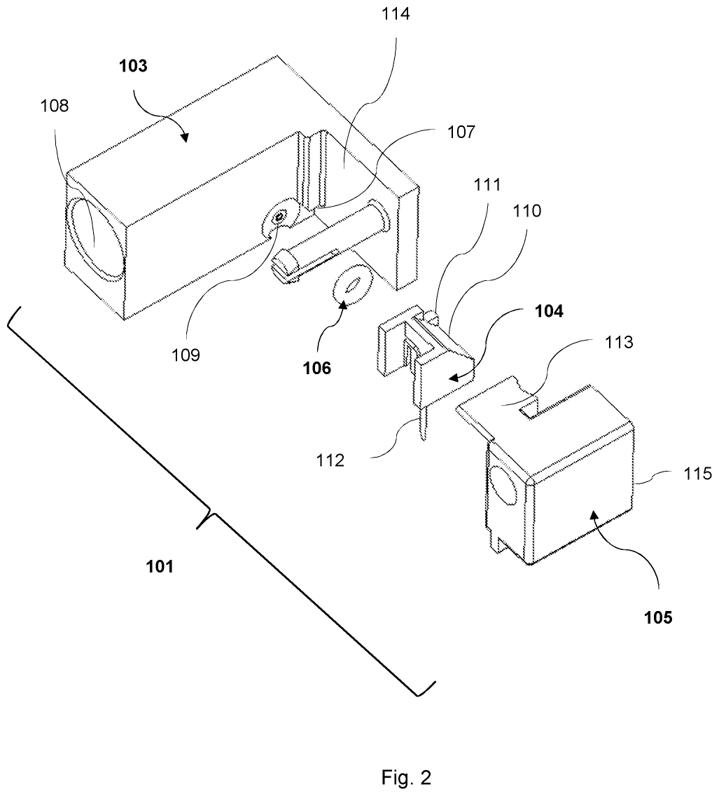

In FIG. 2, an exploded view of a distribution system 101 of a wearable injection device 100 according to an embodiment of the invention is shown. This distribution system 101 can be used as reference in the following explanation of the relationship between the distribution system 101 and the drug-filled container 102. As it can be seen, the distribution system 101 comprises at least a connecting part 103 with an opening 108, which is connecting to the drug-filled container 102 during activation, a delivery part 104 holding a needle 112 for insertion in the user at the injection site, an O-ring 106 for making a tight connection between the connecting part 103 and the delivery part 104, and a transmitting part 105 transmitting an activating force from the user to the delivery part 104 to insert the needle 112.

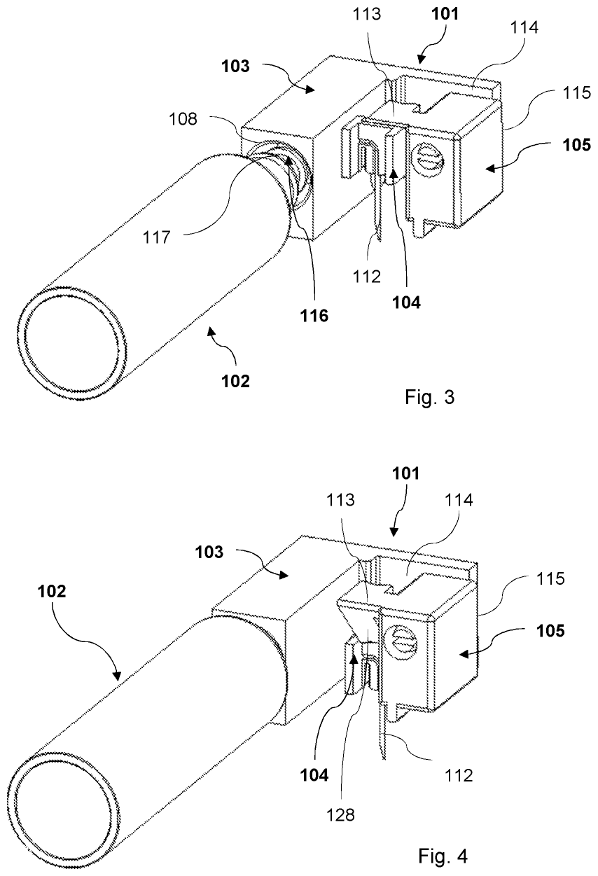

The function of the distribution system 101 and the relation to the drug-filled container 102 with the valve 116 can be seen in FIG. 3. In an initial position, the valve 116 is protruding partly into the opening 108 of the connecting part 103, but the connecting part 103 and the drug-filled container 102 are not yet in fluid communication. A surface 115 of the transmitting part 105 abuts onto a surface 114 of the connecting part 103, and an angled surface 128 (visible in FIG. 4) of a protrusion 113 on the transmitting part 105 abuts onto a corresponding angled surface 110 (visible in FIG. 2) of the delivery part 104. In FIG. 3, the valve 116 can be discerned between the drug-filled container 102 and the connecting part 103 along with an O-ring 117 for securing a tight connection between the drug-filled container 102 and the distribution system 101 during injection. The delivery part 104 is in a retracted position and there is no fluid communication between the connecting part 103 and the delivery part 104 yet.

In FIG. 4, the device 100 has been activated. The user has pushed the flexible push button 127 (see FIG. 1), which in turn has pushed and moved the connecting part 103 and pushed and opened the valve 116 (as will be explained below). The distribution system 101 and the drug-filled container 102 are now in fluid communication. The delivery part 104 is only capable of moving up and down relative to the device 100, and consequently it has been pushed downward by the transmitting part 105, which has been moved by the connecting part 103 because of the abutting surfaces 114, 115. The transmitting part 105 has thereby passed the delivery part 104 and has been locked in this position, and the needle 112 has been inserted at the injection site. A spring (not shown) between the delivery part 104 and the housing of the device 100 has been compressed during the activation, and the delivery part 104 is maintained in this position by a blocking surface 107 of the connecting part 103 resting on a surface 111 on the delivery part 104 (visible in FIG. 2). An inlet hole (not shown) in the delivery part 104 is now aligned with the outlet hole 109 (visible in FIG. 2) in the moving part 103 and, due to the O-ring 106, a tight fluid connection between the connecting part 103 and the delivery part 104 has now been established. This means that, in turn, a tight fluid communication between the drug-filled container 102 and the injection site has been established.

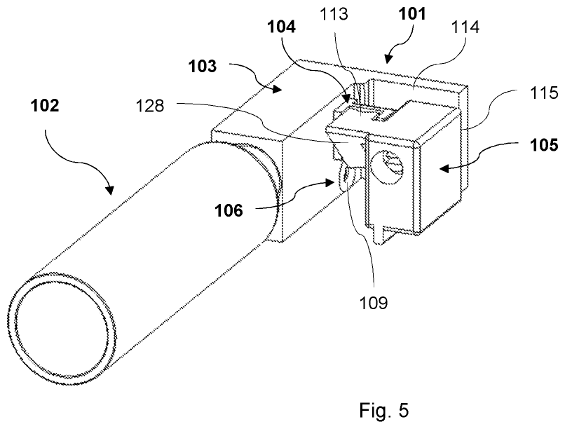

In FIG. 5, the injection has been completed and the valve 116 has caused the connecting part 103 of the distribution system 101 to move a short distance back and away from the drug-filled-container 102 (as will be explained in details below). This has moved the blocking surface 107 of the connecting part 103 away from the surface 111 of the delivery part 104. Because the transmitting part 105 has not moved back along with the connecting part 103, the spring (not shown) compressed during the activation of the device 100 has caused the delivery part 104 to be moved up and the needle 112 to be retracted from the injection site. As can be seen in FIG. 5, the fluid communication between the connecting part 103 and the delivery part 104 has been disconnected.

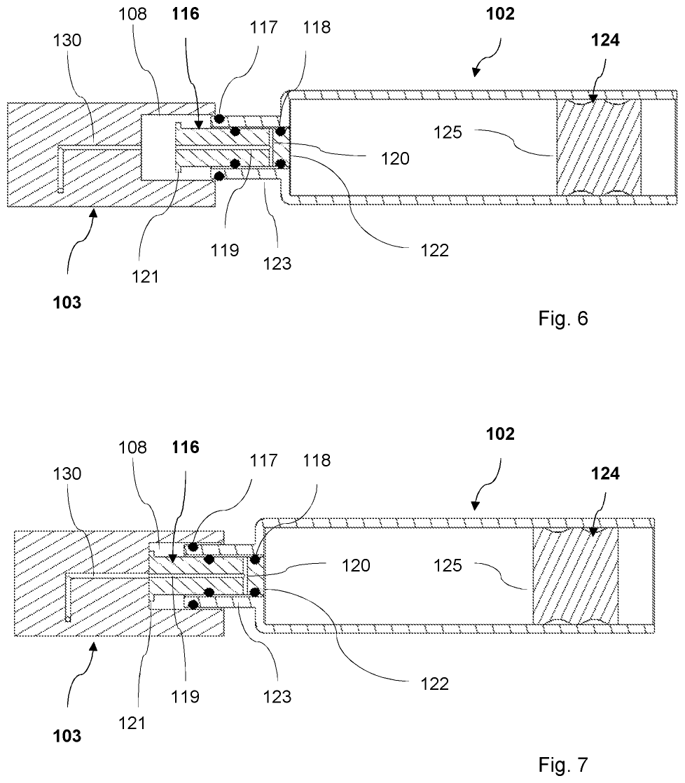

In the following, the function of the valve 116 will be explained with reference to FIGS. 6-9. FIG. 6 is a cross-sectional view of the initial position of the parts before the device 100 is actuated. The valve 116 is initially positioned in an outlet section 123 of the drug-filled container 102. The cylindrical outlet section 123 encloses the openings of a transverse channel 120 connected with an axial channel 119 with an opening pointing away from the container 120, and there is no fluid communication between the drug-filled container 102 and the distribution system 101 in this initial position. An O-ring 118 on the valve 116 ensures a tight separation between the openings of the transverse channel 120 and the drug in the container 102. In FIG. 6, the connecting part 103 has not yet been moved and the O-ring 117 outside the outlet section 123 has not yet made a tight connection between the opening 108 on the connecting part 103 and the drug-filled container 102.

In the following FIG. 7, the connecting part 103 has been moved during an actuation sequence carried out by the user of the device 100. The O-ring 117 is now securing a tight connection between the connecting part 103 (and thereby the distribution system 101) and the drug-filled container 102, and the inlet of a fluid channel 130 in the connecting part 103 is connected to the outlet of the axial fluid channel 119. In other embodiments of the invention, the connecting part 103 may seal to the valve 116 directly.

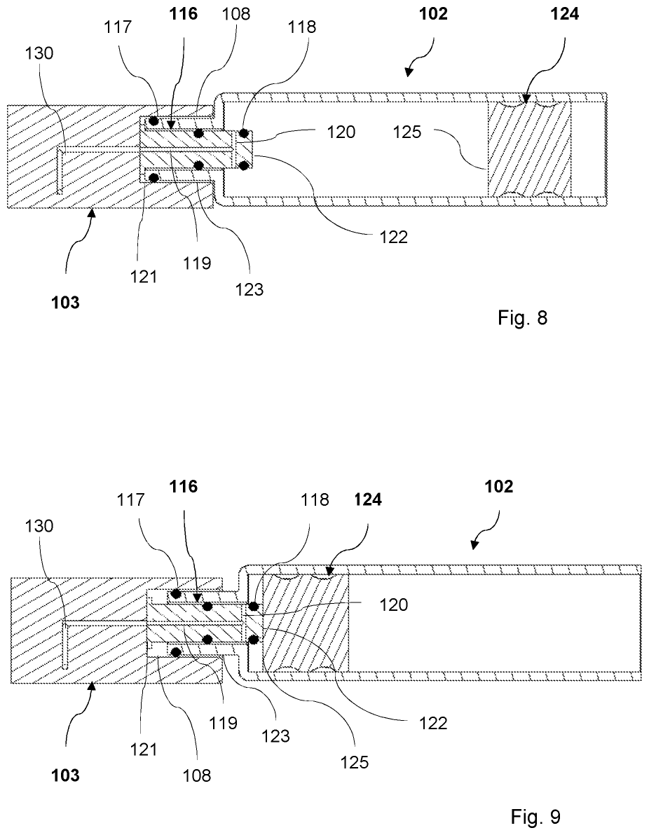

A fully established connection between the connecting part 103 and the drug-filled container 102 can be seen in FIG. 8, wherein a continued movement of the connecting part 103 as part of the activation sequence has moved the valve 116 relative to the drug-filled container 102. This has exposed the openings of the transverse channel 120 to the drug inside the drug-filled container 102, and a tight fluid communication to the distribution system 101 has been established. A flange 121 on the valve 116 stops and controls the longitudinal movement of the valve.

Finally, FIG. 9 shows the scenario wherein the injection has been completed. The plunger 124 has moved to the end of the container 102 causing the content to be expelled, and the plunger surface 125 has abutted onto the valve surface 122 and pushed the valve 116 through a short distance. As described above in relation to FIGS. 3-5, this has caused the valve 116 to move the connection part 103 as well and to set the delivery part 104 free to be moved up, whereby the needle 112 has been retracted to a non-exposed position inside the device 100 and the fluid communication between the connecting part 103 and the delivery part 104 has been disconnected. The plunger 124 movement can either be stopped by the valve 116 or by the continued movement of the plunger against the narrow outlet section 123, or the movement of the connecting part 103 can be adapted to stop the actuator from applying pressure to the plunger 124.

Advantageously, the displacement of the connecting part 103 or an associated part can be used as a tactile signal to inform the user that the injection has been completed or it may initiate a mechanical or electronically generated visual or audible signal. The movement of the connecting part 103 or an associated part in the end of the injection may also be detected electronically and stored and/or transmitted to a server to help health care personnel in assuring that the patient is in compliance.

Instead of the internal channels 119, 120 in the valve 116, external and open channels on the outside or simply smaller diameters on the valve to allow passage of the drug could be envisioned.

In other embodiments, the valve 116 can be configured to be used with a hand-held auto injection, e.g. driven by a spring, and the connecting part 103 may initiate the retraction of the needle 112 or, alternatively, give an acoustic and/or tactile signal to inform the user that the injection has been completed. Such a signal may also be generated by the valve 116 itself or by an intermediate part.

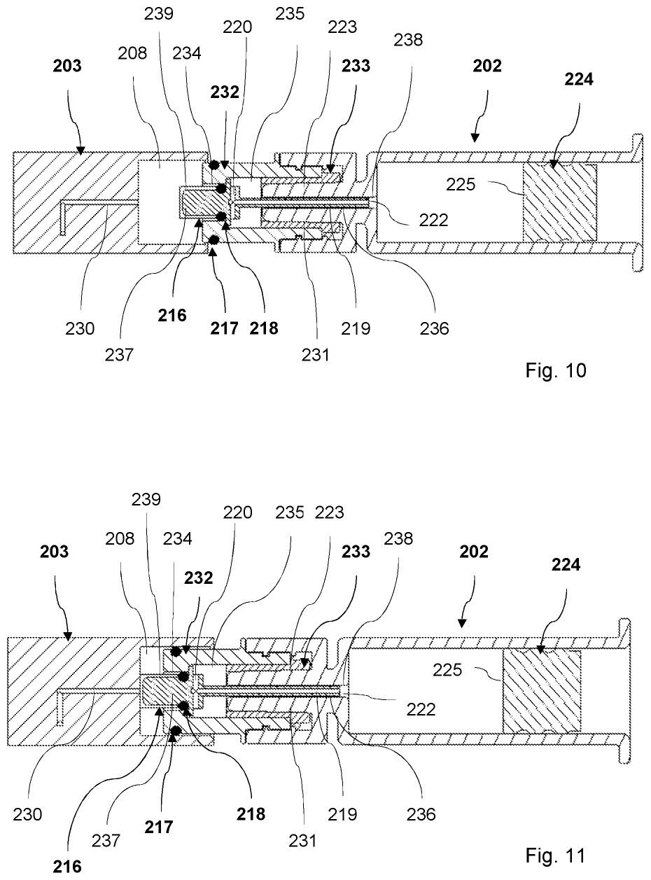

Another embodiment of the invention, which is usable with a standard syringe 202, and which, thereby, is more suitable for existing filling lines is illustrated in FIGS. 10-13. In FIG. 10, a syringe 202 with an outlet section 223 and a luer lock connection 231 or another suitable type of connection is connected with a valve housing 232 via the luer lock. To ensure that the valve housing 232 is tightly connected to the syringe 202, a sealing part 233 of, e.g., silicone is provided between the luer lock surfaces of the syringe 202 and the valve housing 232. The valve housing 232 has a cylindrical opening throughout its length with the same centre axis as the syringe inlet hole 238, which cylindrical opening is divided into an outlet portion 234 with a first diameter near the opening pointing away from the syringe 202 and an inlet portion 235 with a second and larger diameter closer to the syringe 202. A valve 216 has a first portion 236 that protrudes through the syringe inlet hole 238 and partly into the outlet section 223 and has an axial fluid channel 219 extending through the first portion 236 throughout the entire length thereof. A second portion 237 of the valve 216 has a transverse fluid channel 220 perpendicular to and connected with the axial fluid channel 219, and an O-ring 218 is arranged next to the openings of the transverse fluid channel 220. In FIG. 10, the device 100 and, thereby, the valve 216 is in its initial position and the O-ring 218 tightens the outlet portion 234 of the valve housing 232 tightly. The first portion 236 of the valve 216 is enclosed by the syringe inlet hole 238 and the second portion 237 protrudes out of the valve housing 232. A connecting part 203 with an opening 208 and a fluid channel 230, which is part of a drug distribution system 101, is movably arranged relative to the valve housing 232. After activation, the tightness between the opening 208 in the connecting part 203 and the valve housing 232 is ensured by means of an O-ring 217, but in this initial position, the connecting part 203 and the valve housing 232 has not yet connected.

In FIG. 11, the connecting part 203 has been moved a first distance toward the syringe 202 and along the centre axis of the syringe 202 as part of an actuation sequence, whereby a tight connection has been created between the valve housing 232 and the opening 208 in the connecting part 203. The bottom of the opening 208 in the connecting part 203 is now mating with the end of the valve 216.

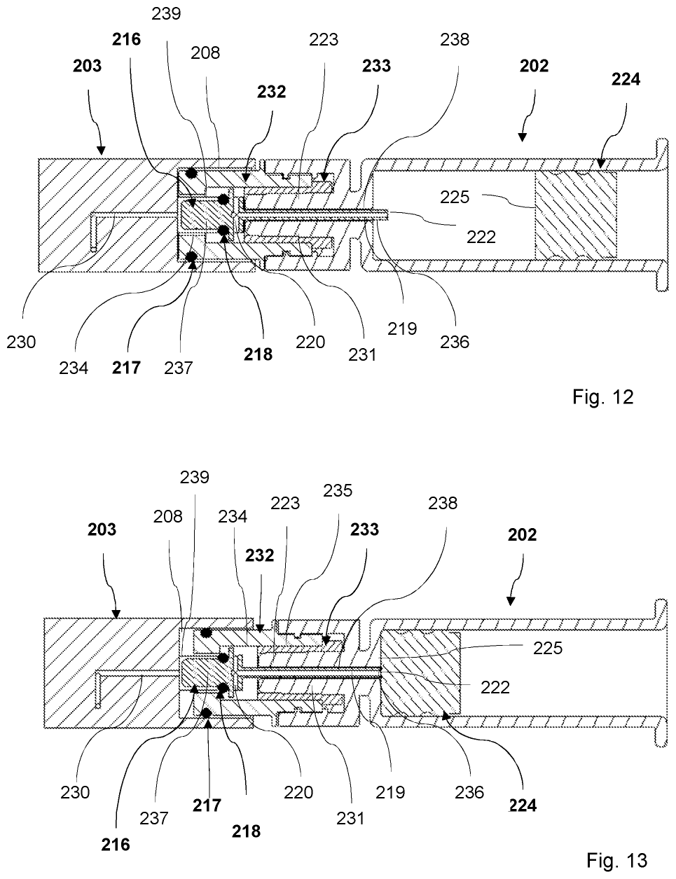

In FIG. 12, further movement of the connecting part 203 has moved the valve 216 a first distance and the O-ring 218 on the valve 216 has been moved into the inlet portion 235 with the larger diameter of the valve housing 232. Drug from the syringe 202 can now flow through the axial fluid channel 219 in the first portion of the valve 216, further through the transverse fluid channel 220 and around the O-ring 218 and the flange on the valve 216 (partly open for passage), through a fluid channel 239 in the valve 216, which is now in connection with the fluid channel 230 in the connecting part 203, and into the distribution system 101. As can be seen, the first portion 236 of the valve 216 is now partly inside the drug-filled volume of the syringe 202, and as the plunger 224 approaches the completion of is movement through the syringe 202 at the completion of the injection, the plunger surface 225 abuts onto the valve surface 222. Further movement of the plunger 224 pushes the valve 216 and the connecting part 203 through a second distance, which is opposite in direction and preferably shorter than the above-mentioned first distance as shown in FIG. 13. The axial fluid channel 219 and the transverse fluid channel 220 can be omitted to obtain a simpler configuration, in which the drug will simply flow along the outside of the first portion 236 of the valve 216.

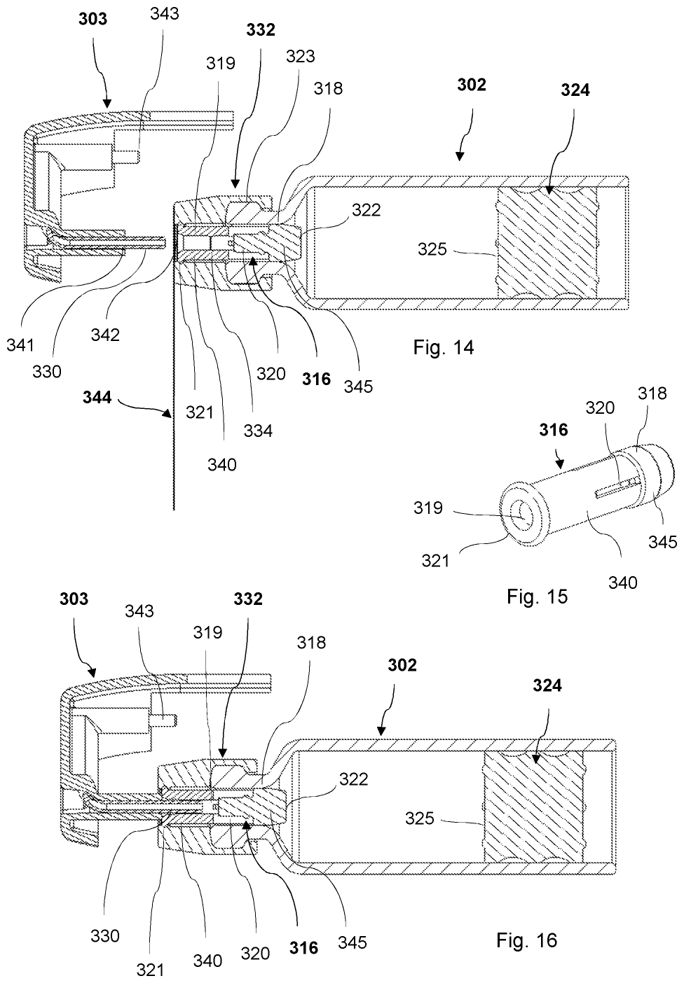

A further embodiment usable with a standard cartridge 302 with a plunger 324, which is normally used in multiple dose injection devices, is illustrated in FIGS. 14 through 17. In FIG. 14, a valve housing 332, which is preferably made of an elastomer, is tightly mounted onto a collar 323 of the cartridge 302 so that a sterile barrier is provided between the two parts. The valve housing 332 has a cylindrical opening throughout its length and in serial connection with inlet hole of the cartridge 302. A valve 316 comprising a valve body 340 and a valve head 345 fixed to each other (as illustrated in FIG. 15) is arranged within the cylindrical opening of the valve housing 332 and fixed in an initial position via a small flange 321 on the valve body 340 arranged in a corresponding groove in the valve housing 332. The valve head 345 protrudes into the opening of the cartridge 302 and the mainly cylindrical surface 318 on the valve head 345 tightly closes the inlet hole of the cartridge 302. A sterile barrier 344 is attached to the end surface of the valve housing 332 to maintain sterility within the connected cylindrical channel 319 and slotted channels 320 of the valve 316 (see FIG. 15) before the device is activated. A connecting part 303 comprises a tube with a first end 330 for connecting with the valve 316 and a second end 343 for connecting with a hypodermic needle (not shown).

In FIG. 16, the sterile barrier 344 has been removed by the user as preparation for the injection, and the connecting part 303 has been moved a first distance as part of the triggering of the device, which has abutted the surface 341 (see FIG. 14) of the connecting part 303 against the surface 342 (see FIG. 14) of the valve body 340. Thereby, the first end 330 of the tube in the connecting part 303 has been moved into the valve body 340 and a tightening recess 334 (see FIG. 14) ensures that fluid from the cartridge 302 can only leave the valve 316 through the opening into the first end 330 of the tube.

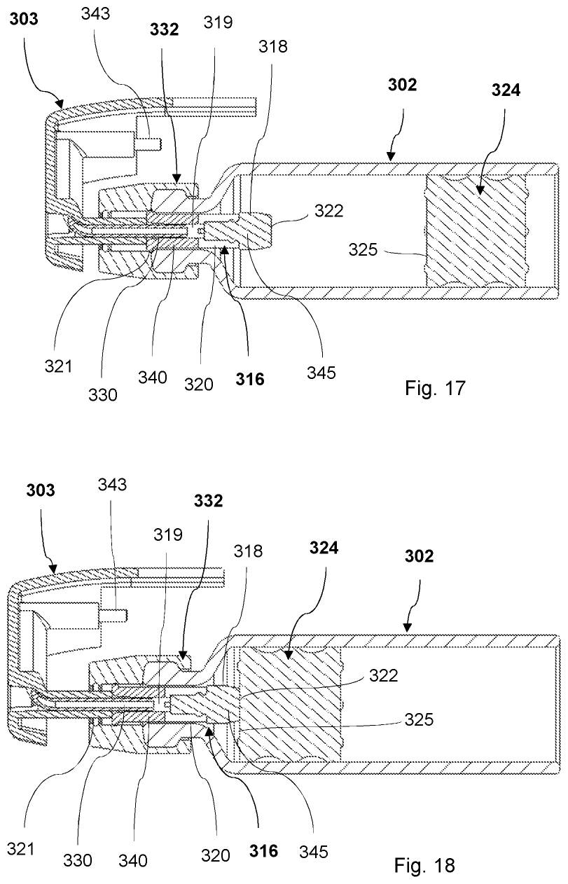

In FIG. 17, the connecting part 303 has been further moved a second distance due to the continued device activation procedure. This has moved the valve 316 within the valve housing 332 and the valve head 345 has been pushed into the cylindrical cavity inside the cartridge 302. The valve 316 is now open so that the fluid from the cartridge 302 can pass through the slotted channels 320 and the cylindrical channel 319 into the opening of the first end 330 of the tube in the connecting part 303. During the movement of the valve 316 and during injection, the small flange 321 on the valve body 340 ensures a tight connection to the cylindrical opening of the valve housing 332 so that fluid cannot leave the cartridge 302 on the outside of the valve body 340.

In FIG. 18, the injection has been fulfilled and the surface 325 of the plunger 324 in the cartridge 302 has abutted the surface 322 of the valve head 345 and pushed the valve 316 and the connecting part 303 a third distance in the opposite direction, the third distance preferably being shorter than the second distance to distinguish the two stages of operation. The slotted channels 320 stay open for the content of the cartridge 302 during and after the movement of the third distance.

The valve head 345 can be made of the same material as the plunger 324 so that the medicament in the cartridge 302 will only be exposed to well-known and proven materials such as glass and rubber, whereby stability of the medicament/device combination is ensured.

### LIST OF REFERENCE NUMBERS

100. Wearable injection device
101. Distribution system
102. Drug-filled container
103. Connecting part
104. Delivery part
105. Transmitting part
106. O-ring
107. Blocking surface of connecting part
108. Opening in connecting part
109. Outlet hole
110. Angled surface of delivery part
111. Surface of delivery part
112. Needle
113. Protrusion on transmitting part
114. First surface of connecting part
115. Surface of transmitting part
116. Valve
117. O-ring
118. O-ring
119. Axial fluid channel
120. Transverse fluid channel
121. Flange on valve
122. Valve surface
123. Outlet section of container
124. Plunger
125. Plunger surface
126. Opening
127. Pushbutton
128. Angled surface of protrusion
130. Fluid channel in connecting part
202. Syringe
203. Connecting part
208. Opening in connecting part
216. Valve
217. O-ring
218. O-ring
219. Axial fluid channel
220. Transverse fluid channel
222. Valve surface
223. Outlet section of syringe
224. Plunger
225. Plunger surface
230. Fluid channel in connecting part
231. Luer lock connection
232. Valve housing
233. Sealing part
234. First diameter of cylindrical opening
235. Second diameter of cylindrical opening
236. First valve portion
237. Second valve portion
238. Syringe inlet hole
239. Fluid channel in valve
302. Cartridge
303. Connecting part
316. Valve
318. Cylindrical surface of valve head
319. Cylindrical channel
320. Slotted channel
321. Flange on valve body
322. Surface of valve head
323. Collar of cartridge
324. Plunger
325. Plunger surface
330. First end of tube in connecting part
332. Valve housing
334. Tightening recess
340. Valve body
341. Surface of connecting part
342. Surface of valve body
343. Second end of tube in connecting part
344. Sterile barrier
345. Valve head

1. A valve for an injection device, which valve is arranged to provide a fluid communication between a drug-filled container and a drug distribution system for distributing a drug to an injection site during use of the device,
wherein the valve is substantially cylindrically shaped and is arranged to be at least partly positioned within an outlet portion of the container so that, in an initial configuration, the valve tightly closes the outlet portion,
wherein the valve is arranged to be moved through a first distance in a first direction partly into the container, whereby fluid communication is established through the outlet portion of the container, and
wherein the valve is further arranged to be moved through a second distance in a second direction opposite of the first direction.

2. The valve according to claim 1, wherein the fluid communication through the outlet portion is established at least partly by means of internal fluid channels through the valve.

3. The valve according to claim 1, wherein the fluid communication through the outlet portion is established at least partly along an outer surface of the valve.

4. The valve according to claim 3, wherein the fluid communication through the outlet portion is established at least partly by means of external and open fluid channels arranged on the outside of the valve.

5. An injection device for subcutaneous injection of a therapeutic drug comprising
a drug-filled container defining a centre axis and comprising an outlet portion and a movable plunger,
a distribution system for distributing the drug to an injection site comprising a connecting part movable along the centre axis, and
a valve according to claim 1, which is at least partly positioned in the outlet portion of the container,
wherein, in an initial configuration, the valve tightly closes the outlet portion of the container,
wherein, during activation of the device, the connecting part causes the valve to move through a first distance in a first direction partly into the container, whereby fluid communication is established through the outlet portion of the container between the container and the distribution system, and
wherein, at the completion of the injection, the plunger in the container pushes the valve through a second distance in a second direction opposite of the first direction, thereby moving the connecting part through the same second distance.

6. The injection device according to claim 5, wherein the second distance is shorter than the first distance.

7. The injection device according to claim 5, wherein the distribution system further comprises
a delivery part with a hypodermic needle for insertion at an injection site at activation of the device, and
a spring means compressed during the insertion,
wherein the connecting part is arranged to maintain the delivery part in an exposed position in relation to the injection device during the injection, and
wherein the connecting part is arranged to release the delivery part to be moved back into a retracted position within the injection device by the compressed spring means, when the connecting part is moved by the valve at the completion of the injection.

8. The injection device according to claim 5, wherein the movement of the connecting part at the completion of the injection is electronically detected.

9. The injection device according to claim 5, wherein, in the initial configuration, the valve is sealed by means of a breakable or removable seal.

10. The injection device according to claim 5, wherein, during activation of the device, the connecting part and the outlet portion of the container are sealed to each other.
